(19) United States
(12) Patent Application Publication
(10) Pub. No.: US 2026/0335588 A1
(43) Pub. Date: Oct. 29, 2026

(54) GLOW-IN-THE-DARK CATHETER PRODUCT

(71) Applicant: Hollister Incorporated, Libertyville, IL (US)

(72) Inventors: Tadas S. Sileika, Lake Forest, IL (US); Vivienne McNulty, Ballina (IE); Darcy R. Balaski, Castlebar (IE); Daniel E. O'Brien, Calry (IE); Evan McGrath, Boyle (IE)

(21) Appl. No.: 19/487,364

(22) PCT Filed: May 22, 2024
(86) PCT No.: PCT/US2024/030547

§ 371 Date: Nov. 24, 2025

Related U.S. Application Data
(60) Provisional application No. 63/504,349, filed on May 25, 2023.

(51) Int. Cl. A61M 25/00; A61L 29/16

(52) U.S. Cl. CPC A61M 25/0017; A61L 29/16; A61L 2300/442

(57) ABSTRACT

A catheter product including a urinary catheter associated with a glow-in-the-dark material. More particularly, a urinary catheter with a glow-in-the-dark material associated with an introducing aid.

The present application claims the benefit of and priority to U.S. Provisional Application No. 63/504,349, filed May 25, 2023, which is hereby incorporated by reference.

### FIELD OF THE INVENTION

This disclosure generally relates to a urinary catheter product. More particularly, this disclosure relates to an intermittent urinary catheter product having a glow-in-the-dark material associated with the product.

### BACKGROUND

Intermittent catheterization is a good option for many users who suffer from various abnormalities of the urinary system. With the advent of intermittent urinary catheters, individuals with problems associated with the urinary system can conveniently self-catheterize to drain the individual's bladder.

Individuals who suffer from urinary incontinence will self-catheterize several times a day. Self-catheterizing individuals may need to self-catheterize in low-light conditions, for instance in the middle of the night. A user may have difficulty seeing a catheter in such low-light conditions.

Additionally, although catheters are typically prepared in sterile environments and are provided with safe handling instructions, catheterizing in low-light conditions may cause a user to mishandle the catheter. The catheter may then become contaminated during use and introduce bacteria into the urethra.

Therefore, there is a need for catheters designed for use in low-light conditions and methods of making and using the same.

### SUMMARY OF INVENTION

There are several aspects of the present subject matter which may be embodied separately or together in the subject matter claimed below. These aspects may be employed alone or in combination with other aspects of the subject matter described herein, and the description of these aspects together is not intended to preclude the use of these aspects separately or the claiming of such aspects separately or in different combination as set forth in the claims appended hereto.

In one aspect a urinary catheter product is provided. The urinary catheter product includes a catheter tube with a catheter tube proximal insertion end and catheter tube distal drainage end. The urinary catheter product also includes an introducing aid. The introducing aid includes a glow-in-the-dark material.

### BRIEF DESCRIPTION OF DRAWINGS

FIG. 1 is a plan view of an embodiment of a urinary catheter of the present disclosure.

FIG. 2 is a perspective view of another embodiment of a urinary catheter of the present disclosure.

### DETAILED DESCRIPTION

A more detailed description of the device in accordance with the present disclosure is set forth below. It should be understood that the description of the specific devices below is intended to be exemplary, and not exhaustive of all possible variations or applications. Thus, the scope of the disclosure is not intended to be limiting and should be understood to encompass variations or embodiments that would occur to persons of ordinary skill.

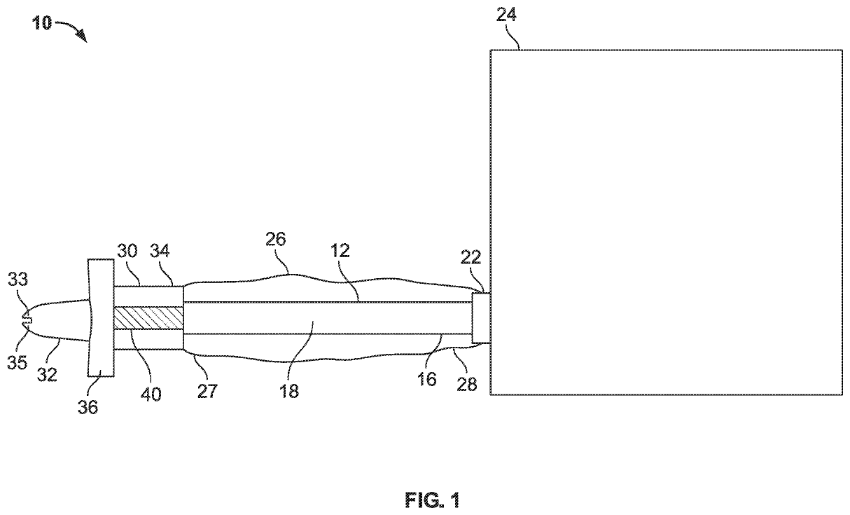

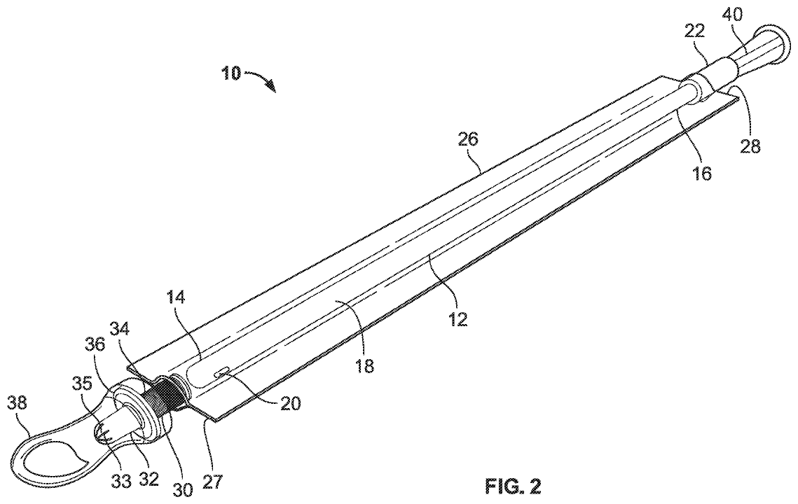

FIGS. 1 and 2 show embodiments of a urinary catheter product 10. The embodiments shown in FIGS. 1 and 2 may include substantially similar features. The same reference numbers will be used to denote a similar feature in each embodiment.

The urinary catheter product 10 may be an intermittent urinary catheter. The urinary catheter product 10 includes a catheter tube 12 having a catheter tube proximal insertion end 14 (shown in FIG. 2) and a catheter tube distal end 16. The catheter tube 12 also includes a tube outer surface 18 and a tube inner surface. The tube inner surface defines a lumen extending from the catheter tube proximal insertion end 14 to the catheter tube distal end 16.

In some embodiments, the urinary catheter tube outer surface 18 may include a hydrophilic polymer surface or coating. The hydrophilic polymer surface or coating may be configured to absorb a hydration medium to lubricate the catheter tube 12 to ease insertion into a urethra. In other embodiments, a gel-lubricant may be applied to catheter tube outer surface 18 to ease insertion.

Additionally, the catheter tube proximal insertion end 14 includes at least one opening 20 (shown in FIG. 2) for receiving urine. In an embodiment, the catheter tube includes more than one opening 20. The at least one opening 20 is in fluid communication with the catheter tube lumen. In an embodiment, the at least one opening 20 for receiving urine may be at least one eyelet. The at least one opening 20 for receiving urine may vary in shape, size, and location without departing from the scope of the disclosure.

Furthermore, the catheter tube distal end 16 includes a drainage opening for draining urine from the lumen. In an embodiment, the catheter tube distal end 16 includes a drainage member 22. The drainage member 22 is in fluid communication with the catheter tube lumen. The drainage member 22 may be, for example, but not limited to, a funnel (shown in FIG. 2) or a drainage member connector for connection to a drainage bag 24 (shown in FIG. 1). Other drainage members known in the art may be used without departing from the scope of the disclosure.

The urinary catheter product 10 may include an introducing aid 30 and a connector 26. The connector 26 may connect the introducing aid 30 to the catheter product 10. The connector 26 includes a connector proximal end 27 and a connector distal end 28. In an embodiment, the connector proximal end 27 may be connected to the introducing aid 30. The connector distal end 28 may be connected to a distal end of the catheter product. For example, the connector 26 may be connected to the catheter tube 12 or to a drainage member 22. The connector 26 may be attached to the introducing aid 30 and catheter tube distal end 16 by, for example, welding, adhesive, or any other suitable manner.

In some embodiments, the connector 26 may be a collapsible sleeve. For example, the sleeve may be a protective or barrier sleeve. The sleeve may have a distal end connected to the distal end of the catheter product 10 at the catheter tube distal end 16 or the sleeve may be connected to a drainage member 22. The sleeve may have a proximal end connected to the proximal end of the catheter product 10 at an introducing aid. The sleeve may define an internal compartment in which the catheter tube 12 may be located. In an embodiment, the sleeve surrounds at least a portion of the catheter tube 12. In another embodiment, the sleeve extends over and surrounds the length of the catheter tube 12.

The sleeve may be made of a flexible material which may be vapor permeable or vapor impermeable, depending on the desired use and packaging. The material of the sleeve may also be liquid impermeable or liquid permeable. The sleeve may be formed of any of a variety of thin, flexible polymeric film materials, such as polyethylene, plasticized PVC, or polypropylene, but elastomeric film materials such as polyurethane, and particularly elastomeric hydrogel materials, may be suitable. In some embodiments, the sleeve may contain a hydration medium.

In another embodiment, the connector 26 can be at least one cord, such as at least one ribbon or string. A proximal end of the cord may be connected to the introducing aid 30 and a distal end of the cord may be connected to the distal end of the catheter product 10. For example, the distal end of the cord may be connected to the catheter tube 12 or to a drainage member 22. The number of cords may vary without departing from the scope of the disclosure.

Introducing aid 30 may include a proximal insertion tip 32 and a distal gripping portion 34. In some embodiments, the introducing aid 30 includes an intermediate flange 36 located in between the proximal insertion tip 32 and the distal gripping portion 34. During use, the intermediate flange 36 may contact the user around the urethral opening and act as a stop to limit the insertion of the insertion tip 32 into the urethra. Additionally, the introducing aid 30 includes an inner surface (not shown) that defines a lumen extending through the insertion tip 32 and distal gripping portion 34. The introducing aid lumen is configured to receive the catheter tube 12 therethrough.

The introducing aid 30 may be connected to the catheter tube 12 by the connector 26. For example, the connector proximal end 27 may be attached to the distal gripping portion 34 of the introducing aid. In some embodiments, the connector may be attached to the intermediate flange 36 or the insertion tip 32.

The proximal insertion tip 32 may be configured to be openable. For example, the insertion tip 32 may be opened by advancing the catheter tube 12 therethrough. In some embodiments, the proximal end of the insertion tip 32 may include one or more slits 33 defining one or more flexible petals 35. The petals 35 may move, bend, and/or resiliently deform from a generally closed configuration to an open configuration when a catheter tube is advanced therethrough. In another embodiment, the insertion tip 32 may include a rupturable proximal end.

The gripping portion 34 includes an opening for receiving the catheter tube 12. The gripping portion 34 may include a textured outer surface to assist a user in gripping or visualizing where to grip the introducing aid 30.

The urinary catheter product 10 may also optionally include a cap 38 (shown in FIG. 2). The cap 38 includes a cavity configured to receive at least the insertion tip 32 of the introducing aid 30. The cap 38 may be attached to the introducing aid 30 via, for example, but not limited to, a friction fit. For example, the cap 38 may be attachable to the intermediate flange 36.

Furthermore, urinary catheter product 10 includes a glow-in-the-dark material 40. The glow-in-the-dark material 40 may assist a user in seeing the urinary catheter product 10 in low-light conditions, for instance, in the dark. The glow-in-the-dark material 40 may illuminate a particular feature or part of the urinary catheter product 10. In some embodiments, the glow-in-the-dark material 40 may include autofluorescent and/or bioluminescent chemicals. For example, the glow-in-the-dark material 40 may include firefly luciferin, coelenterazine, dinoflagellate luciferin, vargulin, bacterial luciferin, cypridina luciferin, green fluorescent protein (GFP), aequorin, renilla luciferin, and gaussian luciferase. Other autofluorescent or bioluminescent chemicals may be used without departing from the scope of the disclosure.

The glow-in-the-dark material 40 may be associated with the urinary catheter product 10. For example, the glow-in-the-dark material 40 may be glow-in-the-dark strips, tritium beads or fibers, and/or fiber optic strands. Other glow-in-the-dark materials may be used without departing from the scope of the disclosure. In some embodiments the glow-in-the-dark material 40 may be attached to the urinary catheter product 10 via, for example, but not limited to, adhesive, welding or any other suitable manner. In other embodiments, the glow-in-the-dark material 40 may be incorporated into the urinary catheter product 10. For example, glow-in-the-dark material such as glow-in-the-dark strips, tritium beads or fibers, and/or fiber optic strands may be embedded into the urinary catheter product 10. For example, the portion of the urinary catheter product 10 including the glow-in-the-dark material may be molded around the material. In other embodiments, the urinary catheter product portion may be heated and/or melted and the glow-in-the-dark material may be pressed into that the portion of the urinary catheter product. In another example, autofluorescent and/or bioluminescent chemicals may be incorporated into the polymer matrix of the parts of the urinary catheter product 10, such as, for example, the introducing aid 30, drainage member 22, connector 26, and/or catheter tube 12. The glow-in-the-dark material 40 may have any color, level of luminance and/or glow pattern. The glow pattern may be any shape and size. In some embodiments, the glow pattern may be an arrow, polygonal or circular shape, letter, words, numbers, etc.

In an embodiment, a urinary catheter product 10 may include a combination of the glow in the dark materials described herein.

In an embodiment, as shown in FIG. 1, the glow-in-the-dark material 40 may be associated with the entire introducing aid 30 or with parts of the introducing aid 30. In other words, at least a portion of the introducing aid 30 includes the glow-in-the-dark material 40. For example, the glow-in-the-dark material 40 may be associated with the distal gripping portion 34, the insertion tip 32, and/or the intermediate flange 36. In an alternative embodiment, only the gripping portion 34 includes glow-in-the-dark material 40. In instances when more than one part of the introducing aid 30 (e.g., the gripping portion 34, the insertion tip 32, and/or the intermediate flange 36) is associated with a glow-in-the-dark material 40, each part may be configured to glow a different color and/or with a different glow shape or pattern. For example, if both the gripping portion 34 and insertion tip 32 are associated with a glow-in-the-dark material 40, the glow-in-the-dark material 40 associated with the gripping portion 34 may be configured to glow a different color than the glow-in-the-dark material 40 associated with the insertion tip 32. In some embodiments, the glow-in-the-dark material 40 associated with the gripping portion 34 may be configured to have a first glow pattern that is different from the glow pattern of the glow-in-the-dark material 40 associated with the insertion tip 32. This may allow a user to see the entire introducing aid 30 in low-light conditions, or it may serve as a visualization of which part of the introducing aid 30 the user should grip.

The glow-in-the-dark material 40 associated with the introducing aid 30 may be any or a combination of the glow-in-the-dark materials 40 described herein, among glow-in-the-dark material known in the art. For example, the glow-in-the-dark material 40 associated with the introducing aid 30 may be at least one glow-in-the-dark strip. Glow-in-the-dark strips may be located on the gripping portion 34, insertion tip 32, and/or intermediate flange 36. The glow-in-the-dark strips may vary in shape and size without departing from the scope of the disclosure. For example, a glow-in-the-dark strip may be sized to cover all or some of a part of the introducing aid (e.g., the gripping portion 34, the insertion tip 32, and/or the intermediate flange 36). In some embodiments, the glow-in-the-dark strip may be wrapped around the outer surface of the introducing aid 30.

In some embodiments, as shown in FIG. 2, the drainage member 22 may include glow-in-the-dark material 40. The glow-in-the-dark material 40 associated with the drainage member 22 may be any or a combination of the glow-in-the-dark materials described herein, among other glow-in-the-dark material known in the art. For example, the glow-in-the-dark material 40 associated with the drainage member 22 may be at least one glow-in-the-dark strip, and/or a glow-in-the-dark material embedded or incorporated in the polymer of the drainage member. The glow-in-the-dark strips may vary in shape and size without departing from the scope of the disclosure. For example, a glow-in-the-dark strip may be sized to cover all or some of the drainage member 22. In an embodiment, the glow-in-the-dark strips may be wrapped around the outer surface of the drainage member 22 or form desired glow pattern, such as the above described shapes.

In some embodiments, the glow-in-the-dark material 40 may be associated with the introducing aid 30, drainage member 22, and/or connector 26 of the urinary catheter product 10. In embodiments where an optional cap 38 is present (as shown in FIG. 2), glow-in-the-dark material 40 may also be associated with the cap 38. In some embodiments, the catheter tube 12 may include a glow-in-the-dark material 40. In embodiments, when more than one part of the urinary catheter product 10 (e.g., the introducing aid 30, the drainage member 22, and/or connector 26) includes glow-in-the-dark material 40, each part may be configured to glow a different color and/or pattern. For example, if the introducing aid 30 and drainage member 22 include glow-in-the-dark material 40, the glow-in-the-dark material 40 associated with the introducing aid 30 may be configured to glow a different color and/or pattern than the glow-in-the-dark material 40 associated with the drainage member 22. Configuring different parts of the urinary catheter product 10 to glow in different colors and/or patterns may provide an indication for the user where to grip/handle the urinary catheter product 10.

To use the urinary catheter product 10, a user may grasp the distal gripping portion 34 of the introducing aid 30 and insert the insertion tip 32 into the urethra. In embodiments where the connector 26 includes a sleeve, the user may then grasp the catheter tube 12 through the sleeve and advance the catheter tube 12 through the introducing aid 30. In embodiments where a sleeve is not present, the user may grasp the drainage member 22 to advance the catheter tube 12 through the introducing aid 30. The catheter tube 12 may be advanced into the and through the urethra until the at least one opening 20 enters the bladder.

It will be understood that the embodiments and examples described above are illustrative of some of the applications of the principles of the present subject matter. Numerous modifications may be made by those skilled in the art without departing from the spirit and scope of the claimed subject matter, including those combinations of features that are individually disclosed or claimed herein. For these reasons, the scope hereof is not limited to the above description but is as set forth in the following claims, and it is understood that claims may be directed to the features hereof, including as combinations of features that are individually disclosed or claimed herein.

1. A urinary catheter product comprising:
a catheter tube with a catheter tube proximal insertion end and a catheter tube distal drainage end; and
an introducing aid, wherein the introducing aid comprises a glow-in-the-dark material.

2. The urinary catheter product of claim 1, wherein the introducing aid comprises a proximal insertion tip and a distal gripping portion.

3. The urinary catheter product of claim 2, wherein the introducing aid further comprises a flange between the proximal insertion tip and distal gripping portion.

4. The urinary catheter product of claim 1, wherein the glow-in-the-dark material comprises autofluorescent and/or bioluminescent chemicals.

5. The urinary catheter product of claim 2, wherein the glow-in-the-dark material is associated with the distal gripping portion.

6. The urinary catheter product of claim 2, wherein the glow-in-the-dark material is associated with the proximal insertion tip.

7. The urinary catheter product of claim 2, wherein the glow-in-the-dark material is associated with the proximal insertion tip and the distal gripping portion, and wherein the glow-in-the-dark material of the proximal insertion tip glows a different color and/or pattern than the glow-in-the-dark material of the distal gripping portion.

8. The urinary catheter product of claim 1, wherein the glow-in-the-dark material is incorporated into the introducing aid.

9. The urinary catheter product of claim 1, wherein the glow-in-the-dark material is attached to the introducing aid.

10. The urinary catheter product of claim 1, wherein the glow-in-the-dark material is at least one glow-in-the-dark strip.

11. The urinary catheter product of claim 1, wherein the glow-in-the-dark material is tritium beads or fibers.

12. The urinary catheter product of claim 1, wherein the glow-in-the-dark material is fiber optic strands.

13. The urinary catheter product of claim 1, further comprising a connector connecting the introducing aid to the catheter tube distal drainage end.

14. The urinary catheter product of claim 13, wherein the connector is a collapsible sleeve defining a compartment to contain at least a portion of the catheter tube, the sleeve having a sleeve proximal end and a sleeve distal end, wherein the sleeve distal end is attached to a catheter tube distal drainage end and the sleeve proximal end is attached to the introducing aid.

15. The urinary catheter product of claim 13, wherein the connector is at least one ribbon or at least one string.

16. The urinary catheter product of claim 13, wherein the connector comprises a glow-in-the-dark material.

17. The urinary catheter product of claim 1, wherein the catheter tube distal drainage end comprises a funnel.

18. The urinary catheter product of claim 17, wherein the funnel comprises a glow-in-the-dark material.

19. The urinary catheter product of claim 18, wherein the glow-in-the-dark material of the funnel glows in a different color and/or glow pattern than the glow-in-the-dark material of the introducing aid.

20. The urinary catheter product of claim 1, wherein the urinary catheter product comprises a cap, wherein the cap comprises a glow-in-the-dark material.

21. (canceled)
